(19) United States
(12) Patent Application Publication
(10) Pub. No.: US 2003/0118597 A1
(43) Pub. Date: Jun. 26, 2003

(54) Process for production of bee venom as pharmaceutical product which can be used effectively in the treatment of rheumatoid arthritis and viral diseases

(76) Inventor: Ali Salem El Abbadi, Giza (EG)

Correspondence Address: NATH & ASSOCIATES, 1030 15th STREET, 6TH FLOOR, WASHINGTON, DC, 20005, US

(21) Appl. No.: 10/268,662

(22) Filed: Oct. 11, 2002

(30) Foreign Application Priority Data
Oct. 23, 2001 (EG) 1127

(51) Int. Cl. A61K 39/38

(52) U.S. Cl. 424/184.1; 119/14.01

(57) ABSTRACT

The traditional treatment for Rheumatoid Arthritis relies on a course of synthetic drugs, which range from the use of gold salts to anti-inflammatory drugs including both steroids and non-steroids. These drugs specially the steroids can affect adrenal and pituitary glands & cause impotence, edema, poor wound healing, reduce neurological response and cardiac irregularities.

VACSERA began the first clinical studies with bee venom therapy and proved its efficiency for treatment of variety of diseases such as Rheumatoid arthritis and viral infections especially (HCV). Bee Venom was separated by a scientific method and then we determine the dosage form, toxicity, bioavailability, teratogenicity, (anti-teratogenic effect), safety and treatment schedule.

The use of Bee venom in treatment of arthritis has been proved to be beneficial to many patients, primarily due to the presence of a number of polypeptides, peptides, enzymes and amines. The venom is administered according to the enclosed leaflet and physician instructions in doses which vary according to the disease and its severity.

### BACKGROUND

### Field of the invention

The invention relates to the adaptability of the traditional medicine (Bee venom) for treatment of a lot of diseases such as rheumatoid arthritis and viral diseases specially (HCV) in a pharmaceutical form and presents a noble service for many patients.

Ancient Egyptians and Babilians were innovators; they used the Bee stings to relief pain accompanying Rheumatism and osteoarthritis since 2000 years B.C.

Chinese have started the bee venom therapy since 1530 In the nineteenth century (1935) French.

Austrian and Russian doctors began the first clinical studies with bee venom therapy by using live Bee stings. They proved its efficiency in treatment of variety of diseases such as Rheumatoid arthritis and viral infections especially (HCV).

### SUMMARY

Nowadays, Apitherapy is practiced by non-professional Bee keepers for treatment of viral infections (specially HCV), bacterial and rheumatic disease,which leads to many side effects such as cellulites due to contamination of the sting pin with microorganism, also beekeepers unawareness of a time schedule for the stings may lead to many serious toxic complications and anaphylactic shock. VACSERA has succeeded in the separation of the venom by certain electrical device. Then all the pre-clinical studies including the dosage form, treatment dose, toxicity, bioavaiolability, teratogenicity, and safety were established.

Then the venom undergoes many purification steps, followed by venom dilution and sterilization through 0.2 micron depth filter sheet. Afterwards, dispensing of the venom takes place after addition of the preservative 0.35% Tricresol in vials with known therapeutic concentration. After that the venom is lyophilized, and is ready for use according to the enclosed leaflet and physician instruction in treatment of viral and rheumatoid arthritis diseases.

### DETAILED DESCRIPTION OF THE INVENTION

Project Goal

Idealistic use of the venom to modulate both immune cells and immune mediators in patients suffering from auto-immune diseases whenever needed.

Getting the Bee venom by a sterile and scientific way and in large amount, which can

huge production scale as a pharmaceutical product.

Manufacturing of the venom locally and no need for importing such product.

The venom undergoes many purification steps and determination of its toxicity is established as preliminary procedure before applying such pharmaceutical product for human use either by injection or using other routes in certain doses according to the severity of the case.

Then the venom is sterilized by using depth filter sheet with porosity 0.2 micron. Afterwards, packaging of the venom takes place after its treatment and mixing with other additives in vials with known therapeutic concentration. After that the venom is lyophilized, and is ready for use according to the enclosed leaflet and physician instructions.

### Description in Details of Venom Extraction

Milking Process:

Venom of bees was obtained by introducing the bees into certain cabinet of glass lined with

metal frames, then a small voltage is applied (from 11-15 volts) during the passage of the bees through the metal frames, this process can stimulate bees to extrude its venom.

Purification process:

After drying of the venom, the plates are removed and washed carefully with sterile physiological saline (NaCl (0.85% )

Centrifugation in a cold centrifuge for 1 hour must be carried out at 4500 r.p.m

Discard the precipitate

The supernatant, is then lyophilized and kept at 4° C. until used.

After lyophilization process, the venom is dissolved in a certain quantity of physiological saline to meet certain concentration/vial

Adjust PH to 7

Addition of preservative (tricresol 0.35%)

Then the solution undergoes sterile filtration through 0.22μ depth filter

Filling

Lyophilization

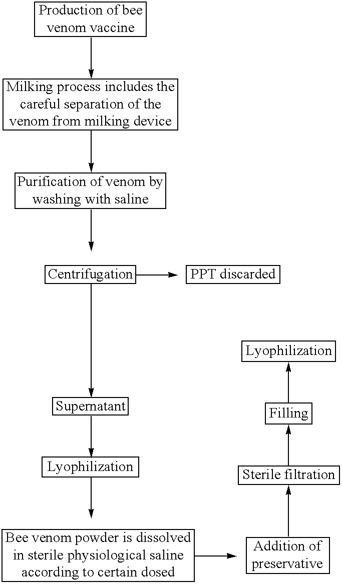

### Pharmacological Properties of the Bee Venom

More than 30 different substances have been characterized in bee venom, the main pharmacological components that reduce inflammation are the high molecular weight peptides including the following: Mellitin, Apamine, Peptide 401(mast cells degranulating peptide), Adolapin and protease inhibitors.

Melittin→Stimulates the hypophyseal adrenal system and releases cortisol that is 100 times more potent than hydrocortisone

Melittin→Stabilizes the lysosomal cell membrane to protect against inflammation and inhibits the complement C₃ system which is involved in the inflammatory process

Ado lapin→Inhibits the microsomal cyclooxygenase system and is 70 times stronger than Indomethacine in animal models, it also inhibits platelet lipooxygenase, which is involved in the production of hydroperoxy-icotetranonic acid and leukotrienes. Also it inhibits thromboxane and prostacycline, which are activated during inflammation.

Protease inhibitors→inhibit carrageenin, prostaglandin E1, bradykinin and histamine-induced inflammations as well as chymotripsin

Also bee venom has strong anti-bacterial, antifungal and radio-protective effect by stimulating the heamopoeitic system.

Bee venom is a strong immunological agent that stimulates the body protective mechanisms against disease.

Chemical properties of bee venom

| | % of | Molecular | Allergenic |
|---|---|---|---|
| Substrate | dry venom | mass (d) | Activity |
| Low molecular weight | <25 | <1.000 | − |
| Histamine | <1 | 111 | − |
| Dopamine | <1 | 153 | − |
| Nor epinephrine | <1 | 169 | − |
| Amino acids | <1 | 100-200 | − |
| Oligo peptides | <14 | 200-1.000 | − |
| Phospholipids | <5 | 100-400 | − |
| Carbohydrates | <2 | <200 | − |
| Peptides | <60 | <10.000 | (+) |
| Melittin | <50 | 2.840 | (+) |
| Apamin | <2 | Tetramer: | − |
| | | 12.500 | |
| Mast cell degranulating | <2 | 2.000 | − |
| peptides (401) | | | |
| Secapin | <0.5 | 2.600 | − |
| tetriapin | <0.1 | 2.000 | − |
| protease inhibitor | <1 | 9.000 | − |
| Prcomine A & B | <2 | 5.00 | − |
| High molecular weight | | >10.000 | (+++) |
| Phosphlipase A | <15 | 16-19.000 | − |
| Phospholipase B | <2 | 22.000 | − |
| Hyaluronidases | <2 | 35-50.000 | − |
| Acid phosphomonoesterase | <2 | 45-90.000 | (+) |
| D-Glucosidase | <1 | Not | ? |
| | | known | |

Bee venom physical characters

Single bee venom volume is 5-20 ul

Venom is colorless, proteinaceous, liquid with sharp bitter taste.

The dry residue is about 12%

Has an aromatic odor

Dried venom has slightly yellowish color

Determination of LD₅₀

Once the venom was obtained, determination of the venom lethal dose was carried out as follows:

Groups of 4 mice (weighing 14-16 gm) were injected in the caudal vein with different concentrations of bee venom

1) The first group was injected with 50 ug of bee venom dissolved in 0.5 ml saline.

2) The second group was injected with 75 ug venom in 0.5 ml saline.

3) The third group was injected with 100 ug venom dissolved in 0.5 ml saline.

4) Finally the 4^{th} group was injected with 150 ug dissolved in the amount of saline as previously mentioned.

### Results

From the previous work, it is clearly demonstrated that, the lethal dose lies between the 3^{rd} and 4^{th} groups, then additional injections with concentrations of 100 ug, 120 ug, and 150 ug (these concentrations lie between 100-150ug) take place, all the previous concentrations caused death in all animal groups. Thus we chose 100 ug as lethal dose which can be identified as the minimum dose of venom that causes death in all animal groups within 24 hrs. and the LD₅₀ between 70-80 ug

The lethal dose is 100 ug

And the LD₅₀ between 70-80 ug

### Determination of the Dosage Form

Our recent work is concerned with the determination of the DOSAGE FORM (either aqueous or haptenuated with complete Freund's adjuvant)of the venom, and testing which is better and more useful for immunization when applying such vaccine treatment for human.

3 groups of rabbits weighing 3.5-4 kg were injected and immunized as follows:]

The first group received 100 ug venom as aqueous preparation, then the schedule was continued for 6 weeks, and before each injection a blood sample is withdrawn to estimate both IgG & IgE levels together with the routine clinical analysis (kidney function, liver function and lipogram tests) to evaluate the effect of the venom on different organs parameters.

The 2^{nd} group received 100 ug venom emulsified with complete freunds adjuvant, and then as previously mentioned as rabbit No. 1

The 3^{rd} group received 50 ug emulsified venom to test the effect of the adjuvant and the extent of amplification of the immune response to bee venom.

### Results

Group No. 1 which is immunized with 100 ug venom as aqueous preparation, exhibit a gradual increase in the IgG level from 660 mg % to 830 mg %, while IgE level decreased to its minimum level of about 19 EU/ml, and no change in the organ functions were observed. (FIGS. 1,2 illustrate our results).

Concerning group No. 2, IgG level increased sharply to a maximum level of about 1250 mg %, while IgE level was fluctuating and reached 78 EU/ml after the 6^{th} venom injection, also no changes in organ functions were observed (FIGS. 3,4 reveal that ).

While the 3^{rd} group which received 50 ug emulsified venom showed an increase in IgG level to its maximum and reached 1250 mg %, whereas IgE level was fluctuating and reached about 63 EU/ml without change in the organ functions (FIGS. 5,6 explain the previous data). RESULTS were expressed as mean±S.D.

From the previous work we can depict that the aqueous preparation can raise the IgG levels gradually and can decrease IgE level to its minimum level in 2 weeks only, on the contrary although the emulsified preparation induces a higher IgG level, it fails to decrease IgE level to its minimum value as compared to the aqueous preparation which means that, the aqueous preparation can induce complete protection (more than emulsified preparation) from any deleterious side effects such as anaphylactic shock when applying such treatment for human trails, also the addition of complete freund's adjuvant causes some abscesses when administered intradermally, due to the composition of the adjuvant itself which contain Tubercle bacilli bacteria that will cause many complications for pre-sensitized patients, also hapten-like adjuvant can increase the incidence of anaphylactic shock.

Some Facts to be Mentioned

The aqueous preparation gives complete protection and decreases the IgE level during 2 weeks post immunization with sustained increase in the IgG level.

Although the administration of the dose as emulsified preparation causes higher increase in the IgG level, we must avoid that form of injection due to the elevation of the IgE level and the abscesses formed during intradermal injection.

### Toxicity

Effect of the Venom on the Renal System

Female Wister rats weighing 150-200 gm were injected intravenously with Africanized bee venom at a dose of 0.4 ul/100 gm of body weight and used in functional and light microscopy studies. The animals were divided into 2 groups: the early group was studied 3-8 hour after inoculation, and the late group was studied 24-30 hours thereafter. The animals showed acute renal failure characterized by reduction of glomerular filtration rate with elevation of plasma creatinine. They also showed increased fractional sodium and potassium excretions, suggesting changes in the proximal portion of the nephron. The water transport through collecting tubules was reduced, with consequent diuresis, indicating functional changes in the distal portion of the nephron. These functional changes were more marked in the early group, with recovery tending to occur after 24 hr, albuminuria was also observed in this group. Light microscopy showed acute tubular necrosis mainly in cortex and outer medulla, with isolated necrosis in cells or small groups of cells and cast formation in the distal and collecting tubules. After 24 hour frequent mitotic figures were found in the tubular epithelium.

The observed acute renal failure was due to acute tubular necrosis which in turn was properly caused by multiple effects, mainly hemodynamic changes secondary to cardio-toxicity and systemic vasodilatation caused by the venom, myohemoglobinuria and the direct action of the venom on tubular cells.

Effect on Cardio-Vascular System

An infarct like myocardial lesions was observed in Wister rats after inoculation of high amount of the venom intravenously.

### Evaluation of Mutagenicity

The mutagenic effect of bee venom was assessed by salmonella/microsome, the venom exerts an antimutagenic effect against the mutagenicity of 4-nitro-phenylenediamine and daunomycin.

Bee venom has an excellent tolerability and wide safety margin up to 700 μg/kg of body weight

Immunotherapy with Bee venom leads to complete protection in more than 98%of the patients with a history of hyperallergy to the venom.

At much higher doses anaphylaxis, pruritis, nettle rash, myxoedema, spasms of the smooth muscles and sudden decrease of blood pressure may develop.

Histamine content of Bee venom at a high dose may cause spasms of coronary vessels

But itching usually shows future good therapeutic results.

Bee venom therapy also leads to increase masculine differentiation.

Immune-therapy during pregnancy did not lead to allergic sensitization of patients' children.

### Bee Venom Pharmacokinetics

Concerning the information that have been requested regarding the pharmacokinetics of bee venom.

First this point is still under research and inexplicit due to multiple components included in the venom (many peptides, polypeptides, enzymes and amino acids ),the molecular weight of the components ranging from 1000 to 90,000 Da, which makes the study lasts more time. However our preliminary data (animal model) elicits that the major allergen component of the venom is Phospholipase A2 and melittin, also the previously mentioned peptides are the most common peptides which induce pharmacological effects together with the Apamin (12,500 Da) of the bee venom, venom fractions of lower molecular weight were pharmacologically inactive.

Nevertheless, toxicokinetics of both classes of venom components were studied

After venom intravenous injection with a dose of( 70 ug/kg) the venom plasma level followed a bi-exponetial decline with distribution half life of 45 min. and an elimination half life of 1.8 hr and the systemic clearance 60 ml/h/kg

Venom level in plasma, after S.C. injection of a dose (700 ug/kg)of venom, increased within a few hours after venom administration to reach a maximum value at 5±0.5 hr. They subsequently followed a monoexponential decline.

### Evaluation of Excretion Route

The route of excretion is determined using intact and nephrectomized rats, after injection of bee venom, the initial 15 min. of the half life was considerably longer in nephrectomized animals after injection, so we concluded that the proximal tubules cells of the kidney participate in the metabolism of circulating venom and higher venom levels persist in plasma of nephrectomized animals. For that reason the immunotherapy with Bee venom is restricted for patients with renal impairment.

### Evaluation of Anti-Bacterial Activity

Also the evaluation of anti-bacterial activity of the bee venom was established by Melittin, the mechanism by which it exerts its action, is not yet clarified but it may be due to the formation of peptide-lipid supramolecular complex pore in the membrane, followed by peptide internalization, simultaneously dissipating the trans-membrane potential and the lipid asymmetry, this also would be of value in developing a more potent antibiotic based on these results.

All the previously mentioned data is due to preliminary study only, and after the completion of this point we will send all the details in.

CLINICAL TRIALS

a) For rheumatoid Arth. Patients

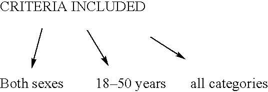

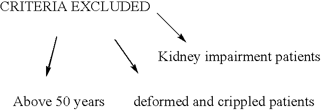

b) For Viral infected patients (HCV infected patients)

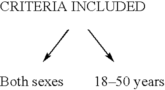

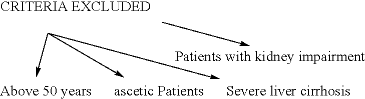

*The product will be tried on 120 volunteer (informed consent) suffering virus infection such as HCV , Rheumatoid arthritis, and bronchial Asthma after detection of the dosage recommended according to physician prescription.
.20 normal volunteers are taken as control

Before application of such therapy, patient must fulfill the following record.

Firstly patient must sign consent form including his agreement to undergo such trials

Personal sheet includes

Sex

Name

Date of birth

Marital status

Address

Phone

Present occupation

Previous occupation

Main Complaint

History of the Previous Illness

Chronic Illnesses Associated

Hypertension

D.M.

Cardiac

Chest

Renal

Liver

Bilharzias

Other

Drug history

Previous occupation

Previous blood transfusion

Investigation Done

Past History

Family History

Medical Examination Report

General examination

Blood pressure

Pulse

Temp.

Appearance

Head &neck

Chest

Abdomen

Limbs

Local Examination

Professional Diagnosis

Investigations Required

Laboratory

Radiology

Others

Diagnosis

Recommendations

Treatment Schedule

Rush schedule

Recommended maintenance schedule (including time interval between injections)

Special Injection Schedule (due to Bernstein et al 1993,1994 and Diaz Gomez et al 1995) was taken and modified according to the type of disease and its severity. This schedule is in conformance with the scientific literature recommendations, which have been published in this field. (Bernstein et al 1993,1994 and Diaz Gomez et al 1995)

Base line analysis included

Liver function tests,

Kidney function tests,

lipogram, and

Fasting blood sugar

to detect the effect of the BEE VENOM on them later on.( for any evidence of toxicity).

### Vacsera Schedule can be Summarized as Follow

Rush Immunization Week

Day 1:

Patient is injected with 25u of the venom solution s.c or i.d. divided on both hands.

Day 3:

Patient is injected with 50 u of the venom solution s.c or i.d. divided on both hands.

Day5:

Patient is injected with 75u of the venom solution s.c or i.d. divided on both hands.

Day 7:

Patient is injected with 100 u of the venom solution s.c or i.d. divided on both hands.

After 2 weeks:

Patient is injected with another 100 u of the venom solution s.c or i.d. but undivided.

Maintenance Treatment

i) For Rheumatic and HCV infected patients

A dose of 100-200 ug can be administered twice weekly for 6 weeks

ii) For hyper-allergic patients against bee venom

Patient is injected monthly with 100 u of the venom for 8 months

Recommendations

Patients having high IgE level must be treated carefully and appropriate precautions are taken.

Analysis recommended for every case is repeated every 21 days to check any evidence of toxicity.

HCV infected Patients, detection of virus removal by PCR are carried out every month to check the rate of viral eradication together with liver function tests.

Rheumatoid arthritis patients are checked permanently for

ESR,

RF-latex

Anti-DNA for sero-negative rheumatoid patients,

ANA,

Interleukin 1 and Interleukin2

besides complete check up for any evidence of toxicity.

Asthmatic patients due to elevation of IgE titer are checked for IgG and IgE every 2 weeks.

Precautions

Divided doses should have time interval 30 min.

Patients with high IgE level should be given Corticosteroids and antiallergic such as Fexofenadin Hcl 180 mg and calcium salts during the days of immunization.

During the rush immunization week, patient stays under observation for 2 hours for any expected deleterious side effects.

Avoid intravenous injection of the venom with huge amount that may lead to infarction like lesions (Animal model trial).

It has no mutagenic effect and does not cross placental barrier

Intravenous injection of the venom can lead to destruction of kidney microtubules.

Applications

Used by individuals, clinics and hospitals as a pharmaceutical biological product

either inject able according to certain immunization schedules to modulate

immune cells and immune mediators and eradicates viral infection

Or in other forms like cream, lotion, and plasters to treat a lot of diseases.

We claim

1- The venom extraction method (milking process).

2- Method of claim 1, in which the bees undergo milking process several times with no decrease in bees number.

3- The method of purification, by which the venom is obtained in the most pure form.

4- The therapeutic benefits which have been established for every fraction of the 33 fractions of the venom.

5- According to claim 4,where in the Melittin can achieve its therapeutic effect by stimulation of hypophyseal-adrenal gland to release cortisol and stabilizes the lysosomal cell membrane against inflammation and inhibits the complement C3 system that is involved in the inflammation process.

6- According to claim 4,where, Adolapin inhibits microsomal cyclooxygenase system and also inhibits thromboxane and prostacycline, which are activated during inflammation.

7- According to claim 4,where, protease inhibitors inhibit carrageenin, prostaglandin E₁, bradykinin and histamine induced inflammation as well as chymotripsin.

8- The administration method including both rush and maintenance treatment.

9- A process according to claim 8, where the week of rush injection is recommended for patient desensitization against any future adverse effects, in which ascending doses are administered from 25 to 100 μg.

10- A process according to claim 8, including maintenance dose of about 100-200 μg can be administered twice weekly for rheumatoid arthritis & viral infected patients.

11- A process according to claim 8, in which patients who exhibit hypersensitivity against bee venom are desensitized by using 100 μg of the venom for 8 months as monthly injected dose

12- A process of claim 8,in which the duration of the treatment is not less than 8 consecutive weeks.

13- The recommended dosage form of the venom, whereas the administration of aqueous preparation raises IgG level gradually and lowers IgE level to its minimum value in two weeks only, reveals dual benefits in preventing the main cause of anaphylactic shock during the maintenance treatment and avoiding sudden elevation of IgG against the venom due to the immune tolerance that originated as a result of administration of allergen in high doses with narrow time intervals.
